(19) United States
(12) Patent Application Publication
(10) Pub. No.: US 2005/0070603 A1
(43) Pub. Date: Mar. 31, 2005

(54) Use of glycerides to alleviate symptoms of digestive disorders

(76) Inventors: Praful K. Shah, Superior, CO (US); Karen L. Russell, Superior, CO (US)

Correspondence Address: Madhavi C. Chander, 5342 Desert Mountain Court, Boulder, CO, 80301, US

(21) Appl. No.: 10/951,204

(22) Filed: Sep. 27, 2004

Related U.S. Application Data
(60) Provisional application No. 60/505,830, filed on Sep. 25, 2003.

(51) Int. Cl. A61K 31/225

(52) U.S. Cl. 514/547

(57) ABSTRACT

Disclosed is a method of treatment for alleviating pain and other problems associated with digestive disorders in general and Irritable Bowel Syndrome in particular using glycerin fatty acid esters such as medium and long chain glycerides.

### FIELD OF THE INVENTION

The present invention relates to the field of treatment of symptoms associated with digestive disorders and more specifically to the treatment of Irritable Bowel Syndrome using glycerin fatty acid esters, long and medium chain glycerides.

### BACKGROUND OF INVENTION

Irritable Bowel Syndrome (here after IBS) is the most common of all gastrointestinal disorders, affecting 11-14% of adults and accounting for more than 50% of all patients with digestive complaints (G. Triadafilopoulos et al., Bowel Dysfunction in Fibromyalgia, Digestive Dis. Sci. 36 (1): 59-64 (1991); W. G. Thompson, Irritable Bowel Syndrome: Pathogenesis and Management, Lancet 341: 1568-72 (1993)). It is thought that only a minority of people with IBS actually seek medical treatment. Patients with IBS present disparate symptoms, for example, abdominal pain predominantly related to defecation, alternating diarrhea and constipation, abdominal distention, gas, and excessive mucus in the stool. Often IBS is diagnosed by ‘exclusion’ rather than any definitive test.

A number of possible causes for IBS have been proposed, but none has been fully accepted (W. G. Thompson, 1993). These hypotheses included a fiber-poor Western diet, intestinal motility malfunction, abnormal pain perception, abnormal psychology or behavior, psycho physiological response to stress.

A high fiber diet increases stool bulk and shortens gut transmit time. However the presence of IBS in non-Western countries, such as China and India, and the failure of dietary fiber supplements to treat IBS in double-blind clinical trials are inconsistent with the “fiber hypotheses” for causing IBS. (W. Bi-zhen and P. Qi-Ying, Functional Bowel Disorders in Apparently Healthy Chinese People, Chin. J. Epidemiol. 9:345-49 (1988); K. W. Heaton, Role of Dietary Fibre in IBS. In: R. W. Read (ed), Irritable Bowel Syndrome, Grune and Stratton, London, pp. 203-22 (1985); W. G. Thompson et al., Functional Bowel Disorders and Functional Abdominal Pain, Gastroenterol. Int. 5:75-92 (1992)).

Those experiencing chronic IBS pain are often depressed and anxious. Treatment with tricyclic antidepressants and serotonin antagonists has been used to raise the pain threshold and treat associated anxiety of some IBS patients. (U.S. Pat. Nos. 5,063,245, 5,861,398 and 5,612,366).

Because there has been no known underlying cause for IBS, treatment of IBS has been primarily directed to symptoms of pain, constipation or diarrhea symptoms. For example, administration of the polypeptide hormone relaxin, used to relax the involuntary muscles of the intestines, is a treatment taught to relieve the pain associated with IBS (U.S. Pat. No. 5,863,552).

### OBJECTS OF THE INVENTION

The present invention relates to a method of treatment to alleviate the symptoms associated with IBS comprising an effective amount of a glycerin fatty acid ester or a mixture of glycerin fatty acid esters in a pharmaceutically acceptable carrier.

One aspect of the present invention relates to a method of treatment to alleviate the symptoms of IBS with minimal side effects comprising an effective amount of a glycerin fatty acid ester or a mixture of glycerin fatty acid esters as an additive to food or drink.

Another aspect of the present invention relates to a method of treatment to alleviate the symptoms of IBS comprising a monoglyceride, a diglyceride or a triglyceride or a mixture containing any or all of these glycerides in a pharmaceutically acceptable carrier.

According to the present invention, the glycerin fatty acid esters useful in the treatment of IBS have the following chemical structure: 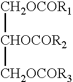
where R₁, R₂ and R₃ are each H, an alkyl, olefinic or aromatic with at least one R being an alkyl, olefinic or aromatic group.

### DETAILED DESCRIPTION OF THE INVENTION

The present invention relates to the use of glycerin fatty acid esters for relieving symptoms of pain and discomfort associated with IBS and related digestive disorders.

Accordingly, an embodiment of the present invention is to provide a method of treatment to alleviate symptoms associated with IBS and other related digestive disorders comprising an effective amount of a glycerin fatty acid ester or a mixture of these esters, in a pharmaceutically acceptable carrier.

According to the present invention, a glycerin fatty acid ester useful in alleviating the symptoms of IBS has the following chemical structure: 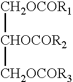
where R₁, R₂ and R₃ are each H, an alkyl, olefinic or aromatic with at least one R being an alkyl, olefinic or aromatic group; the R group may be branched or linear having 1 to 20 carbon atoms.

When only one R is an alkyl or olefinic or aromatic group, it is a monoglyceride, when two R groups are an alkyl, olefinic or aromatic groups (identical or different) it is a diglyceride and finally when all the three R groups are an alkyl, olefinic or aromatic groups (identical or different) it is a triglyceride.

When the R of a triglyceride is an alkyl group and is between 6-12 carbons, the triglyceride is a medium chain glyceride (hereafter MCT) and when the R of a triglyceride is an alkyl group and is more than 12 carbons, the triglyceride is a long chain glyceride (hereafter LCT).

MCTs are widely used in parenteral nutrition, baby formulas, medical foods, drug products (as excipients), energy supplements and other food products. MCTs are considered GRAS (Generally Recognized As Safe) material (A. H. Kibbe, Handbook of Pharmaceutical Excipients, 3^{rd} Ed., (2000)). A review of article by Traul et al., summarizes the toxicological properties of MCT (K. A. Traul et al., Review of the Toxicologic Properties of MCTs., Food Chem. Toxicol. 38 (1):79-98, (2000)).

The present invention provides a method of treatment for relieving symptoms of pain and discomfort associated with IBS. Fats and oils are known to have strong stimulus of colonic contraction and would aggravate intestinal discomfort. Thus use of glycerin fatty acid esters for relief of IBS symptoms is counter-intuitive. These compounds may be administered orally to the digestive tract directly or formulated as an emulsion or suspension with an emulsifier or a surfactant. For example, a glyceride (or a mixture of these) may be mixed with milk, cream, yogurt, soymilk and/or any other dairy or non-dairy food products. Other forms of administration of a glyceride (or a mixture of the glycerides) can be in the form of a nutritional drink, a food bar, a powdered beverage, a capsule or a tablet to be given orally, or dosage forms to be given by other routes of administration, for example, an injectable emulsion or a topical lotion.

Accordingly, the present invention has been described with some degree of particularity directed to the exemplary embodiments of the present invention. Since many possible embodiments may be made of the invention without departing from the spirit and scope thereof, it is to be understood that all matter herein set forth or shown in the accompanying example is to be interpreted as illustrative and not in a limiting sense.

MCT oil, Neobee 895 was obtained from Stepan Food Ingredients (Maywood, N.J.). It is also known as Captrin or Caprylic triglyceride, and its chemical structure is as follows:

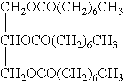

### EXAMPLE

A patient suffering from IBS for over 25 years with symptoms such as pain associated with intestinal gas, bloating and constipation has reported complete relief of the symptoms after taking one tablespoon (approx. 15 mL) of MCT (Neobee 895 from Stepan Company) per day. The patient noticed some improvement initially after taking one teaspoon (approx. 5 mL) of MCT as an emulsion in soymilk (approx. 240 mL) per day. Over a period of 8 weeks, the patient increased the dose of MCT to approx. 15 mL (in approx. 240 mL of soymilk) per day to achieve complete symptomatic relief from IBS.

The use of MCT was continued without any adverse effects and the patient continued to have complete relief from the IBS symptoms (approx. 1 year). There has been no noticeable change observed in the HDL, LDL and triglyceride levels after 10 months of use of MCT.

1. A method of treating the symptoms associated with irritable bowl syndrome or other related digestive disorders, comprising administering an effective amount of a glycerin fatty acid ester or its mixtures to a patient in need thereof.

2. A method according to claim 1, wherein the glycerin fatty acid is represented by the formula: 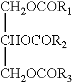
where R1, R2 and R3 can be H, an alkyl, an alkenyl or an aromatic group with at least one R being an alkyl, an alkenyl or an aromatic group.

3. A method according to claim 2, wherein the said alkyl group is branched or linear having 3-20 carbon atoms.

4. A method according to claim 2, wherein the said alkyl group is branched or linear having 6-12 carbon atoms.

5. A pharmaceutical composition for the treatment of symptoms associated with irritable bowl syndrome or other related digestive disorders, comprising an effective amount of a glycerin fatty acid ester or its mixtures, in a pharmaceutically acceptable carrier suitable for administration to a patient in need thereof.

6. A pharmaceutical composition according to claim 5, wherein the glycerin fatty acid is represented by the formula: 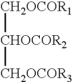
where R1, R2 and R3 can be H, an alkyl, an alkenyl or an aromatic group with at least one R being an alkyl, an alkenyl or an aromatic group.

7. A pharmaceutical composition according to claim 6, wherein the said alkyl group is branched or linear having 3-20 carbon atoms.

8. A pharmaceutical composition according to claim 6, wherein the said alkyl group is branched or linear having 6-12 carbon atoms.

9. A pharmaceutical composition according to claim 5, wherein the glycerin fatty acid ester or its mixtures can be administered in the form of a suspension, gel, tablet, capsule, nutritional drink, food bar, powdered beverage for oral administration or an injectable solution, emulsion or suspension or a topical lotion, gel or patch.
